(19) United States
(12) Patent Application Publication
(10) Pub. No.: US 2011/0319626 A1
(43) Pub. Date: Dec. 29, 2011

(54) Use of Atorvastatin Lactols as Medicaments

(75) Inventors: Derek Lindsay, Wigan (GB); Peter Jackson, Wigan (GB)

(73) Assignee: BRADFORD PHARMA LIMITED, Wigan (GB)

(21) Appl. No.: 13/255,707

(22) PCT Filed: Mar. 10, 2010
(86) PCT No.: PCT/GB2010/050408

§ 371 Date: Sep. 9, 2011

(30) Foreign Application Priority Data
Mar. 10, 2009 (GB) 0904102.1

(51) Int. Cl. C07D 401/14; C07D 405/06

(52) U.S. Cl. 546/279.1; 548/517

(57) ABSTRACT

This invention relates to the discovery of novel atorvastatin analogues. More specifically, the invention relates to novel atorvastatin analogues which have utility in treating conditions treatable by the inhibition of HMG-CoA reductase.

The present invention relates to atorvastatin lactols. In particular, the present invention relates to the use of atorvastatin lactols in the manufacture of a medicament for treating certain conditions. Conditions that are treatable using the compounds of the present invention include conditions which are modulated by the enzyme 3-hydroxy-3-methylglutaryl-coenzyme A reductase (HMG-CoA reductase). Inhibition of the enzyme therefore represents a viable therapy for a number of diseases. The compounds used in the invention are 6-(3- or 4-carboxamido-substituted pyrrol-1-yl)-4-hyroxy-3,5-dihydro-pyran-2-ol derivatives.

Trans-6-[2-(3- or 4-carboxamido-substituted pyrrol-1-yl)alkyl]-4-hydroxypyran-2-one compounds are lactones which were first disclosed in U.S. Pat. No. 4,681,893. This document also disclosed their corresponding ring opened acid equivalents. The lactones apparently do not have intrinsic activity of their own. However, the corresponding ring-opened acid equivalents are useful as cholesterol biosynthesis inhibitors. Also disclosed in U.S. Pat. No. 4,681,893 are various methods of manufacture for such compounds.

Atorvastatin, which is the R form of the ring-opened acid of trans-5-(4-fluorophenyl)-2-(1-methylethyl)-N,4-diphenyl-1-[2-tetrahydro-4-hydroxy-6-oxo-2H-pyran-2-yl)ethyl]-1H-pyrrole-3-carboxamide, and its use in the inhibition of the biosynthesis of cholesterol was first disclosed in EP 0409281. Atorvastatin both in racemic form, and in the form of its [R—(R*,R*)] isomer is a potent inhibitor of HMG-CoA enzyme.

Clin Invest Med, Volume 24, No 5, p 258-72, 2001 (Baker and Tamopolsky) discloses that whilst statins having an open, hydroxy acid conformation are active, the lactone, closed-ring analogue is inactive. Hepatic hydrolysis at alkaline pH decyclises and hence activates the lactone prodrugs lovastatin and simvastatin in vivo. However, one problem with such compounds is that extensive first path metabolism leads to rapid clearance of these statins.

Similarly, Trends in Pharmacological Sciences, Volume 19, Issue 1, 1 Jan. 1998, Pages 26-37 discloses that the inactive lactones must be metabolised to their corresponding open hydroxy acid forms in order to inhibit HMG-CoA reductase.

The lactone form, and also the ring opened active form, may suffer problems in terms of stability over an extended period of time. This represents a significant problem during manufacture of an active principal or during extended storage of the same in a pharmacy. For example, loss of the hydroxy group in a dehydration reaction may occur. The resulting decomposition product may have a double bond that is conjugated with the lactone carbonyl group and this may tend to favour formation of the decomposition product. Equally, in the ring opened form, one of the possible decomposition products could also have a conjugated double bond with the acid carbonyl group.

It is therefore an aim of the present invention to provide compounds capable of inhibiting HMG-CoA reductase. Atorvastatin is a very potent inhibitor of HMG-CoA reductase. It is also therefore an aim of the present invention to provide compounds capable of inhibiting HMG-CoA reductase which have an IC50 value comparable to or better than that of atorvastatin. Ideally, these compounds will have good stability and bioavailability relative to atorvastatin. It is thus an aim to provide compounds having improved stability. Ideally, the compounds will have an extended shelf-life. It is thus an aim of the present invention to provide compounds capable of inhibiting HMG-CoA reductase which have increased half-life. It is thus an aim of the present invention to provide further compounds capable of inhibiting HMG-CoA reductase and having improved bioavailability. It is also an aim of the present invention to provide compounds capable of inhibiting HMG-CoA reductase and increasing promotion of high density lipoprotein (HDL). It is also an aim of the present invention to provide compounds capable of reducing low density lipoprotein (LDL) and increasing promotion of high density lipoprotein (HDL). Specifically, it is an aim of the present invention to provide compounds capable of reducing low density lipoprotein (LDL) and increasing promotion of high density lipoprotein (HDL) by more than 10%, preferably up to 15% or higher. The invention thus seeks to provide therapies for inhibiting cholesterol biosynthesis. The invention also aims to treat a range of diseases in which cholesterol formation is inhibited.

This invention provides compounds that achieve one or more of the above aims.

According to one aspect, the present invention provides a use of a compound of Formula I and pharmaceutically acceptable salts and solvates thereof:

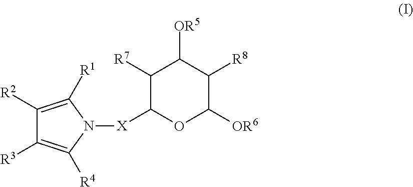

in the manufacture of a medicament for treating a condition which is modulated by the enzyme 3-hydroxy-3-methylglutaryl-coenzyme A reductase (HMG-CoA reductase), wherein:

R¹, R⁴ and one of R² and R³ are independently selected from the group comprising: hydrogen, halo, C₁₋₆ alkyl, C₂₋₆ alkenyl, C₃₋₆ cycloalkyl, aryl, C₁₋₄ alkyl aryl, heterocyclyl, and C₁₋₄ alkyl heteroaryl;

the other of R² and R³ is —CONR⁹R¹⁰ where R⁹ and R¹⁰ are independently selected from the group comprising: hydrogen, C₁₋₆ alkyl, C₂₋₆ alkenyl, aryl, C₁₋₄ alkyl aryl, heteroaryl, C₁₋₄ heteroaryl;

R⁵ and R⁶ are independently selected from the group comprising: hydrogen, C₁₋₆ alkyl, C₁₋₆ haloalkyl, C₂₋₆ alkenyl, C₃₋₆ cycloalkyl, aryl, C₁₋₆ alkyl aryl, C₁₋₆ alkanoyl aryl, heteroaryl, C₁₋₆ alkanoyl heteroaryl, and C₁₋₆ alkyl heteroaryl; provided always that both R⁵ and R⁶ are not hydrogen;

R⁷ and R⁸ are independently selected from the group comprising: H, C₁₋₄ alkyl and halo;

X is —(CR^{a}R^{b})ₘ(CR^{a}═CR^{b})ₙ(CR^{a}R^{b})ₒ where R^{a} and R^{b} are independently selected from the group comprising: H, methyl, ethyl and halo and m, n, and o are independently 0, 1, 2, or 3 provided that m+n+o is not more than 3; and wherein

each of the above groups R¹ to R¹⁰ may, where chemically possible, be independently optionally substituted by from 1 to 5 groups chosen independently at each occurrence from the groups comprising: halo, C₁₋₃ alkyl, halo C₁₋₃ alkyl, C₁₋₃ alkoxy, C₁₋₃ haloalkoxy, hydroxy, and cyano.

Usually conditions that are modulated by HMG-CoA reductase are conditions that would be treated by the inhibition of the enzyme using a compound of the present invention.

According to another aspect, the present invention provides a compound of Formula I and pharmaceutically acceptable salts and solvates thereof:

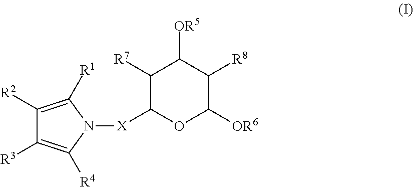

for use in treating a condition treatable by the inhibition of the enzyme 3-hydroxy-3-methylglutaryl-coenzyme A reductase (HMG-CoA reductase) wherein R¹-R¹⁰, R^{a}, R^{b}, X, m, n and o are as defined above.

According to another aspect, the present invention provides a method of treating a condition treatable by the inhibition of the enzyme 3-hydroxy-3-methylglutaryl-coenzyme A reductase (HMG-CoA reductase) comprising administering an effective amount of a compound of Formula I:

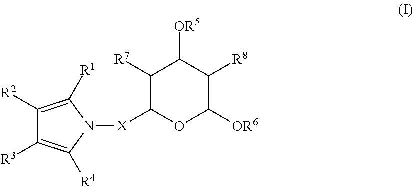

or a pharmaceutically acceptable salt or solvate thereof, wherein R¹-R¹⁰, R^{a}, R^{b}, X, m, n and o are as defined above.

Compounds have activity in their own right or may in certain cases ring open under physiological conditions to corresponding compounds having inhibitory activity.

Pharmaceutically acceptable salts of the compounds of formula (1) include the acid addition and base salts thereof.

Suitable acid addition salts are formed from acids which form non-toxic salts. Examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 1,5-naphthalenedisulfonate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulphate and hemicalcium salts. For a review on suitable salts, see “Handbook of Pharmaceutical Salts Properties, Selection, and Use” by Stahl and Wermuth (Wiley-VCH, Weinheim, Germany, 2002).

Pharmaceutically acceptable salts of compounds of formula (1) may be prepared by one or more of three methods:
(i) by reacting the compound of formula (1) with the desired acid or base;
(ii) by removing an acid- or base-labile protecting group from a suitable precursor of the compound of formula (1) or by ring-opening a suitable cyclic precursor, for example, a lactone or lactam, using the desired acid or base; or
(iii) by converting one salt of the compound of formula (1) to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionisation in the resulting salt may vary from completely ionised to almost non-ionised.

The compounds of the invention may exist in both unsolvated and solvated forms. The term ‘solvate’ is used herein to describe a molecular complex comprising the compound of the invention and a stoichiometric amount of one or more pharmaceutically acceptable solvent molecules, for example, ethanol. The term ‘hydrate’ is employed when said solvent is water.

Included within the scope of the invention are complexes such as clathrates, drug-host inclusion complexes wherein, in contrast to the aforementioned solvates, the drug and host are present in stoichiometric or non-stoichiometric amounts. Also included are complexes of the drug containing two or more organic and/or inorganic components which may be in stoichiometric or non-stoichiometric amounts. The resulting complexes may be ionised, partially ionised, or non-ionised. For a review of such complexes, see J Pharm Sci, 64 (8), 1269-1288 by Haleblian (August 1975).

Hereinafter all references to compounds of formula (1) include references to salts, solvates and complexes thereof and to solvates and complexes of salts thereof.

The compounds of the invention include compounds of formula (1) as hereinbefore defined, including all polymorphs and crystal habits thereof, prodrugs and isomers thereof (including optical, geometric and tautomeric isomers) as hereinafter defined and isotopically-labeled compounds of formula (1).

Before purification, the compounds of the present invention may exist as a mixture of enantiomers depending on the synthetic procedure used. For example, the compounds of the present invention may exist as a mixture of enantiomers having a ratio of between 2:1 and 3:1, though they may also occur in other ratios. The enantiomers can be separated by conventional techniques known in the art. Thus the invention covers individual enantiomers as well as mixtures thereof. When the chemical structures disclosed herein includes an ‘*’, it is intended that the compound is a mixture of enantiomers having a ratio of between 2:1 and 3:1.

For some of the steps of the process of preparation of the compounds of formula (1), it may be necessary to protect potential reactive functions that are not wished to react, and to cleave said protecting groups in consequence. In such a case, any compatible protecting radical can be used. In particular methods of protection and deprotection such as those described by T. W. GREENE (Protective Groups in Organic Synthesis, A. Wiley-Interscience Publication, 1981) or by P. J. Kocienski (Protecting groups, Georg Thieme Verlag, 1994), can be used. All of the above reactions and the preparations of novel starting materials used in the preceding methods are conventional and appropriate reagents and reaction conditions for their performance or preparation as well as procedures for isolating the desired products will be well-known to those skilled in the art with reference to literature precedents and the examples and preparations hereto.

Also, the compounds of formula (1) as well as intermediate for the preparation thereof can be purified according to various well-known methods, such as for example crystallization or chromatography.

In an embodiment, R¹ is selected from the group comprising: hydrogen, C₁₋₆ alkyl, C₂₋₆ alkenyl or C₃₋₆ cycloalkyl. In an embodiment, R¹ is selected from the group comprising: C₂₋₆ alkenyl or C₃₋₆ cycloalkyl. In an alternative embodiment, R¹ is C₁₋₆ alkyl. In an embodiment, R¹ is methyl, ethyl, propyl or butyl. In an embodiment, R¹ is i-propyl.

In an embodiment, R² is —CONR⁹R¹⁰.

In an embodiment, R³ is selected from the group comprising: aryl, C₁₋₄ alkyl aryl, heteroaryl and C₁₋₄ alkyl heteroaryl. In an embodiment, R³ is selected from the group comprising: aryl and C₁₋₄ alkyl aryl. In an embodiment, R³ is aryl. In an embodiment, R³ is phenyl.

In an embodiment, R⁴ is selected from the group comprising: aryl, C₁₋₄ alkyl aryl, heteroaryl and C₁₋₄ alkyl heteroaryl, wherein each of the aforementioned groups may be optionally substituted as discussed above in relation to the first aspect. In an embodiment, R⁴ is selected from the group comprising: aryl and C₁₋₄ alkyl aryl. In an embodiment, R⁴ is aryl. In an embodiment, R⁴ is phenyl. In an embodiment, R⁴ is substituted with halo, optionally wherein the halo is fluorine. In an embodiment, R⁴ is 4-fluorophenyl.

In an embodiment, R⁵ is selected from the group comprising: hydrogen, C₁₋₆ alkyl, aryl, C₁₋₄ alkyl aryl, heteroaryl and C₁₋₄ alkyl heteroaryl. In an embodiment, R⁵ is selected from the group comprising: hydrogen, C₁₋₄ alkyl aryl and C₁₋₄ alkyl heteroaryl. In an embodiment, R⁵ is C₁₋₆ alkanoyl heteroaryl, e.g. methanoyl heteroaryl. In a preferred embodiment, R⁵ is methanoyl pyridyl, e.g. 2-methanolyl pyridine, 3-methanolyl pyridine or 4-methanolyl pyridine, preferably 3-methanolyl pyridine. In an embodiment, R⁵ is hydrogen. In an alternative embodiment, R⁵ is C₁₋₄ alkyl aryl, e.g. —C₁ alkyl-Ph, —C₂ alkyl-Ph, —C₃ alkyl-Ph, or —C₄ alkyl-Ph. In an embodiment, R⁵ is benzyl.

In an embodiment, R⁶ is the same as R⁵.

In a further embodiment, R⁶ is selected from the group comprising: C₁₋₆ alkyl, C₂₋₆ alkenyl, C₃₋₆ cycloalkyl and aryl. In a preferred embodiment, R⁶ is C₁₋₆ alkyl. In further preferred embodiment, R⁶ is methyl, ethyl, n-propyl, i-propyl or t-butyl. In another preferred embodiment, R⁶ is C₂₋₆ alkenyl. In another preferred embodiment, R⁶ is allyl. In another preferred embodiment, R⁶ is C₃₋₆ cycloalkyl. In another preferred embodiment, R⁶ is cyclohexyl. In another embodiment, R⁶ is aryl, such as optionally substituted phenyl.

In an embodiment, R⁷ is H.

In an embodiment, R⁸ is H.

In an embodiment, R⁹ is hydrogen or C₁₋₄ alkyl. In an embodiment, R⁹ is hydrogen.

In an embodiment, R¹⁰ is selected from the group comprising: aryl, C₁₋₄ alkyl aryl, heteroaryl and C₁₋₄ alkyl heteroaryl. In an embodiment, R¹⁰ is selected from the group comprising: aryl and C₁₋₄ alkyl aryl. In an alternative embodiment, R¹⁰ is selected from the group comprising: heteroaryl and C₁₋₄ alkyl heteroaryl. In an embodiment, R¹⁰ is phenyl.

In an embodiment, n is 0. In an embodiment, m=1, n=0 and o=1 or m=2, n=0 and o=0 or m=0, n=0 and o=2. In an alternative embodiment, m=3, n=0 and o=0. In an alternative embodiment, m=1, n=0 and o=0. In an alternative embodiment, m=1, n=1 and o=0, or m=0, n=1 and o=1.

In an embodiment, R^{a} is H at each occurrence.

In an embodiment, R^{b} is H at each occurrence.

In a further embodiment, each R^{a} is H, each R^{b} is H and m=2, n=0 and o=0.

Aryl groups include aromatic ring systems comprising 6, 7, 8, 9, 10, 11, 12, 13, 14, 15 or 16 ring carbon atoms. Aryl groups may consist of a single ring but may include a polycyclic ring system, having two or more rings, at least one of which is aromatic. Aryl groups include: phenyl, naphthyl, fluorenyl, azulenyl, indenyl and anthryl groups.

In an embodiment, the aryl group is phenyl.

Heteroaryl groups include aromatic heterocyclic ring systems having 5, 6, 7, 8, 9, 10, 11, 12, 13, 14, 15 or 16 ring atoms with 1 to 4 heteroatoms independently selected from nitrogen, oxygen and sulfur. The group may be a polycyclic ring system, having two or more rings, at least one of which is aromatic, but is more often monocyclic. Preferred heteroaryl groups are monocyclic groups containing 5 or 6 ring atoms. Heteroaryl groups include: pyrrolyl, pyrazolyl, imidazolyl, pyrazinyl, oxazolyl, isoxazolyl, thiazolyl, furyl, thiophenyl, pyridyl, pyrimidyl, benzimidazolyl, indolyl, isoquinolyl, quinoxalinyl and quinolyl.

In an embodiment, the heteroaryl group is selected from the group comprising: pyridine, pyrimidine, pyrazine, pyrazole, and oxazole. Preferably the heteroaryl group is pyridine.

When one or more of the above groups is optionally substituted, each optional substituent is preferably an independently chosen halo atom. Amongst halo, chloro and fluoro are preferred. Preferably, the halo atoms are the same when there are more than one.

In an embodiment, R¹ is C₁₋₄alkyl, preferably i-propyl, and R⁴ is optionally substituted aryl, preferably 4-fluorophenyl.

In another embodiment, R² is —CONR⁹R¹⁰ wherein R⁹ is optionally substituted aryl, preferably phenyl; R¹⁰ is hydrogen; and R³ is optionally substituted aryl, preferably phenyl.

In a further embodiment, R¹ is C₁₋₄alkyl, preferably i-propyl; R² is —CONR⁹R¹⁰ wherein R⁹ is optionally substituted aryl, preferably phenyl, R¹⁰ is hydrogen; R³ is optionally substituted aryl, preferably phenyl; and R⁴ is optionally substituted aryl, preferably 4-fluorophenyl.

The relationship between the groups R⁵ and R⁶ is important for the activity of the compounds. Thus both R⁵ and R⁶ cannot be hydrogen. In one embodiment, R⁵ is not hydrogen. In one embodiment, R⁶ is not hydrogen.

In another embodiment, R⁵ is optionally substituted benzyl and R⁶ is optionally substituted C₁₋₆ alkyl. In this embodiment, preferably the alkyl group is propyl or butyl, preferably isopropyl or tertbutyl. In another embodiment, R⁵ is optionally substituted benzyl and R⁶ is optionally substituted C₂₋₆ alkenyl. In this embodiment, preferably the alkenyl group is allyl.

In another embodiment, R⁵ is H and R⁶ is optionally substituted C₁₋₆ alkyl. In this embodiment, preferably the alkyl group is propyl. In another embodiment, R⁵ is H and R⁶ is C₃₋₆ cycloalkyl. In this embodiment, preferably the cycloalkyl group is cyclohexyl.

In another embodiment, R⁵ is H and R⁶ is optionally substituted aryl. In this embodiment, preferably the optionally substituted aryl is optionally substituted phenyl, e.g. phenyl substituted by alkoxy (e.g. methoxy).

In another embodiment, R⁵ is C₁₋₆ alkanoyl heteroaryl and R⁶ is optionally substituted C₁₋₆ alkyl. In this embodiment, the alkyl group is preferably methyl and the C₁₋₆ alkanoyl heteroaryl group is preferably methanoyl heteroaryl, (e.g. methanoyl pyridine).

In an embodiment, the compound has a structure selected from:

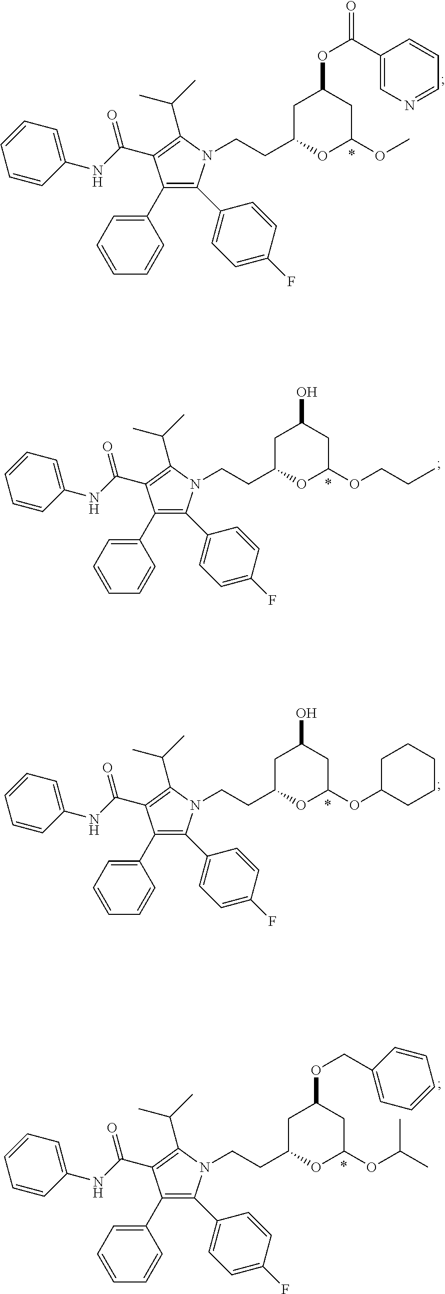

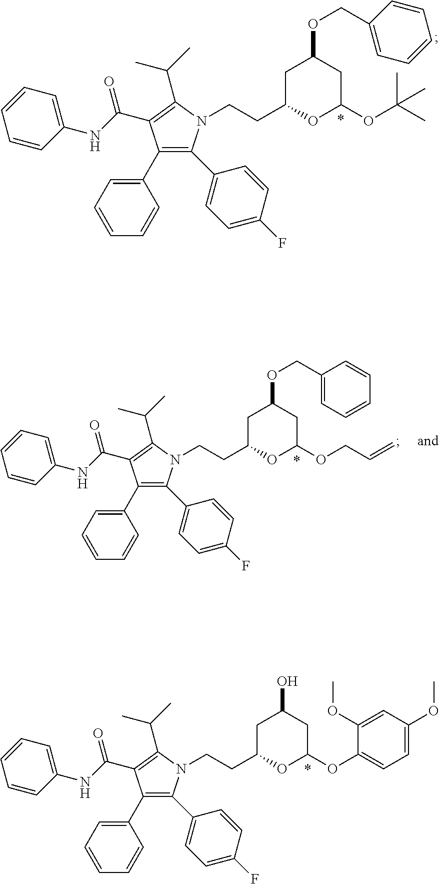

As mentioned above, statins having an open, hydroxy acid conformation are known to have an inhibitory effect on HMG-CoA reductase. It is also known that the lactone, closed-ring analogue of such hydroxy acids are inactive with respect to inhibiting HMG-CoA reductase and that decyclisation of the lactone is necessary to activate the lactone. However, we have found that functionalised lactols of the present invention have a significant inhibitory effect on HMG-CoA reductase in their own right. This is surprising in view of the fact that these molecules are conformationally constrained in ring closed form.

Examples of conditions that may be treated by the inhibition of HMG-CoA reductase include hypercholesterolemia, atherosclerosis and hyperlipidemia. Statins have been used in the secondary prevention of cardiovascular disease, or in the primary prevention of cardiovascular disease when the risk for cardiovascular disease is significantly raised. It is therefore expected that the compound of the present invention will have utility in the treatment or prevention of cardiovascular diseases due to their inhibitory activity. Example cardiovascular diseases which may be treatable by the compounds of the present invention include: coronary heart disease, myocardial infarction, stroke and peripheral artery disease. In addition, these compounds may also have a beneficial effect in the treatment of inflammation, dementia, cancer, nuclear cataracts, diabetes and hypertension.

The conditions that may be treated by the inhibition of HMG-CoA reductase may be a condition of the human or animal body. These compounds are intended in particular for human patients.

The atorvastatin derivatives of the present invention can be assayed using the following procedure in which the plasma triglyceride level is measured after treating a rat with a compound of the present invention (or atorvastatin). The change in rat plasma triglyceride levels is considered to be a fair test for determining HMG CoA reductase activity.

The procedure used is as follows: male SD rats (Harlan) are housed in groups of 6 under a 12 h light dark cycle (lights on 07.00 h) with free access to food (normal laboratory chow) and water. Animals between 148-183 g are allocated to treatment groups of 8 balanced by body weight and treatments are balanced across cages.

Solutions including 5 mg/mL of the atorvastatin analogues (in e.g. 10% PEG300/10% cremophor/80% methyl cellulose (0.5%)) and a suspension including 5 mg/kg of atorvastatin (formulated in 0.5% Tween in 0.5% methyl cellulose) are made.

The rat subjects are orally dosed with one of the atorvastatin analogues (25 mg/kg) or atorvastatin (25 mg/kg po), BID for 3 or 5 days.

Sixteen hours after the last treatment, terminal plasma samples are taken, stored at −20° C., and transported on dry ice for analysis of triglyceride levels.

Data for each time-point are analysed by 1-way ANOVA and post-hoc Dunnett's test.

Processes for the manufacture of the compounds of the present invention are disclosed in WO2005/012246, in particular, in the examples. The disclosure of WO2005/012246 insofar as the synthetic procedures are concerned forms part of the disclosure of the present invention. In the interests of brevity, the details of these synthetic procedures is not reproduced here but it is intended that this subject matter is specifically incorporated into the disclosure of this document by reference.

The present invention also includes the synthesis of all pharmaceutically acceptable isotopically-labelled compounds of formula (I) wherein one or more atoms are replaced by atoms having the same atomic number, but an atomic mass or mass number different from the atomic mass or mass number usually found in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds, for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹O, ¹⁸F, ¹⁵O and ¹³N, can be useful in Positron Emission Topography (PET) studies for examining substrate receptor occupancy.

Isotopically-labelled compounds can generally be prepared by conventional techniques known to those skilled in the art or by processes analogous to those described using an appropriate isotopically-labelled reagent in place of the non-labelled reagent previously employed.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

### General Procedure

All assays were carried out in a reaction buffer containing 100 nM KₓPO₄ at pH 7.2, 1 mM EDTA, 500 mM KCl and 1 mg/ml BSA. The concentrations of NADPH and HMG-CoA were both 200 μM. The enzyme concentration used is unknown although this concentration is 10-fold lower than that of the stock solution purchased. Inhibitors were dissolved in 75% DMSO. Where inhibitors were found to be insoluble or only partly soluble in 75% DMSO, 100% DMSO was used. Reactions were activated by the addition of enzyme and agitated for 12 seconds following the addition. Absorbance readings were then taken every 20 seconds for 600 seconds. In initial tests the concentration of each inhibitor was set at 50 nM to identify which compounds were the better inhibitors, compared to the known Pravastatin inhibitor. After these were identified, assays were carried out varying their concentrations from 0 nM to 50 nM allowing IC50 values to be calculated.

### EXAMPLE 1

The following procedure was followed using a HMG-CoA Reductase assay kit obtained from Sigma-Aldrich (catalogue number CS1090). The assay is based on the spectrophotometric measurement of the decrease in absorbance at 340 nm of NADPH in solution. A decrease in absorbance is caused by the oxidation of NADPH by the catalytic subunit of HMGR in the presence of the substrate HMG-CoA. Effective inhibition of the HMG-CoA leads to a reduction in oxidation of NADPH which in turn leads to a smaller reduction in the absorbance at 340 nm over time. This is illustrated in the following reaction scheme:

HMG-CoA+2NADPH+2H⁺→mevalonate+2NADP⁺+CoA-SH

Compounds showing the best inhibitory action are those which reduce the absorbance least.

### Preparation of the Assay Solution

Ultrapure water (17 MΩ-cm or equivalent was used for the preparation of reagents and throughout the procedure.

First, an assay buffer solution was prepared using the following method: 0.2 ml of assay buffer, 5× (catalogue number A5981) was diluted with 0.8 ml of ultrapure water. The resulting buffer solution was kept on ice or stored at −20° C. for further use.

Next, 25 mg of NADPH (catalogue number N6505) was reconstituted with 1.5 ml of the buffer solution. The reconstituted NADPH was stored in working aliquots at −20° C.

The HMG-CoA substrate solution (catalogue number S7447), HMG-CoA reductase (catalogue number H8789) and inhibitor solution (e.g. pravastatin, catalogue number I5909) were kept on ice throughout the procedure.

1. Before beginning, the spectrophotometer was set at 37° C. and 340 nm, with a kinetic programme: 1 ml sample, read every 20 seconds for up to 10 minutes.

2. The appropriate volumes of the reaction solutions were added according to Table 1 (1 ml assay).

TABLE 1
Reaction volumes for 1 ml samples

| | 1x Assay | Test compound/ | | | |
|---|---|---|---|---|---|
| Sample | buffer | Pravastatin | NADPH | HMG-CoA | HGMG |
| Blank | 920 μl | — | 20 μl | 60 μl | — |
| Activity | 915 μl | — | 20 μl | 60 μl | 5 μl |
| Inhibition | 910 μl | 5 μl | 20 μl | 60 μl | 5 μl |

The reagents were added to the reaction in the following order:
a. Add a buffer to all samples.
b. Add the inhibitor (test compound/Pravastatin) to the inhibition sample.
c. Add the reconstituted NADPH to all samples.
d. Add Substrate Solution (HMG-CoA) to all samples.
e. Add HMG-CoA Reductase (HMGR) to the Activity and Inhibition samples.
f. Mix the samples thoroughly.

3. The kinetics programme was started immediately. The activity of the product was calculated according to the following equation:

$$Units \text{/} mg P = \frac{( Δ {A}_{340} / {min}_{sample} - Δ {A}_{340} / {min}_{control} ) \times TV}{12.44 \times V \times 0.6 \times LP}$$

where:
12.44=ε^{mM}−the extinction coefficient for NADPH at 340 nm is 6.22 mM⁻¹cm⁻¹. 12.44 represents the 2 NADPH consumed in the reaction.
TV=total volume of the reaction in ml (1 ml for cuvettes)
V=volume of enzyme used in the assay (ml)
0.6=enzyme concentration in mg-protein (mgP0/ml (0.55-0.65 mgP/ml)
LP=light path in cm (1 for cuvettes).

### EXAMPLE 2

The following table provides IC50 values for particular atorvastatin compounds of the present invention.

| Compound Structure | IC₅₀ (nM) |
|---|---|
| 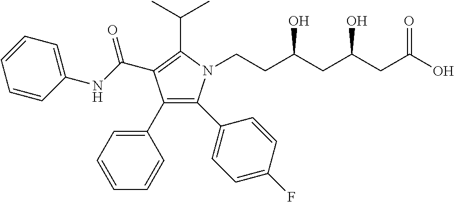 | 7 |
| 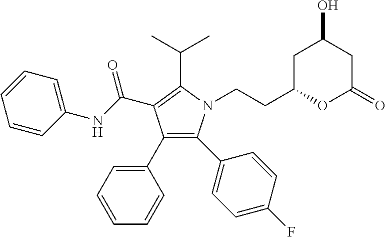 | 3 |
| 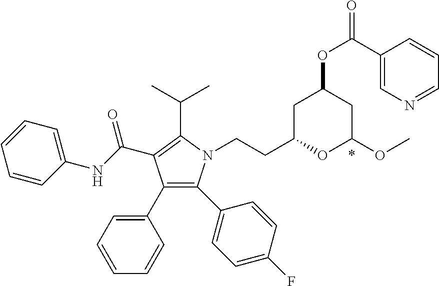 | <1 |
| 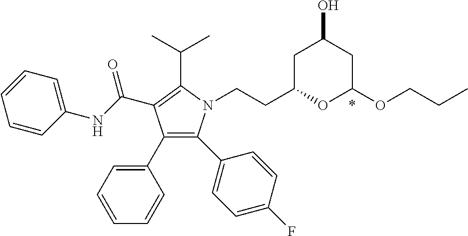 | <1 |
| 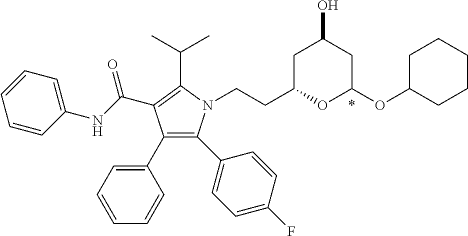 | <1 |
| 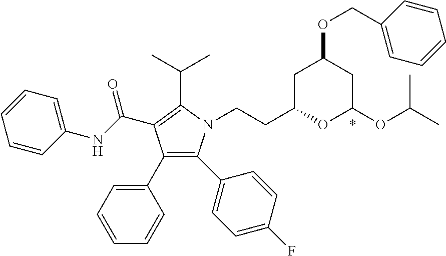 | 4 |
| 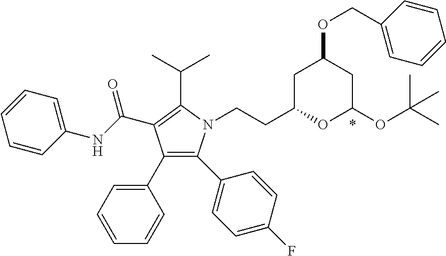 | 3 |
| 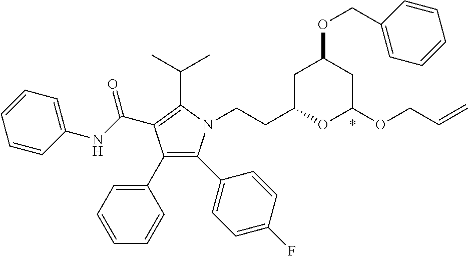 | 1 |
| 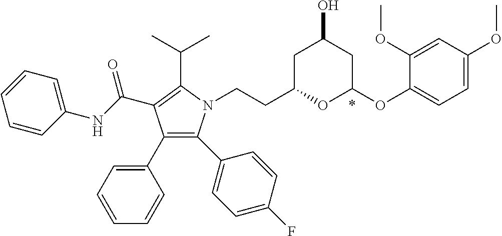 | <1 |

1. A compound of Formula I and pharmaceutically acceptable salts and solvates thereof: 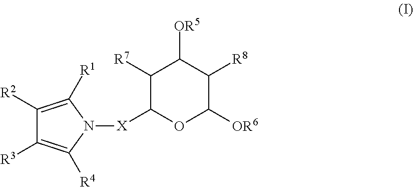
for use in treating a condition treatable by the inhibition of the enzyme 3-hydroxy-3-methylglutaryl-coenzyme A reductase (HMG-CoA reductase), wherein the condition treatable by the inhibition of HMG-CoA reductase is selected from the group consisting of: hypercholesterolemia, atherosclerosis, hyperlipidemia, cardiovascular disease, coronary heart disease, myocardial infarction, stroke, peripheral artery disease, inflammation, dementia, cancer, nuclear cataracts, diabetes and hypertension;
further wherein:
R¹, R⁴ and one of R² and R³ are independently selected from the group consisting of: hydrogen, halo, C₁₋₆ alkyl, C₂₋₆ alkenyl, C₃₋₆ cycloalkyl, aryl, C₁₋₄ alkyl aryl, heterocyclyl, and C₁₋₄ alkyl heteroaryl;
the other of R² and R³ is —CONR⁹R¹⁰ where R⁹ and R¹⁰ are independently selected from the group consisting of: hydrogen, C₁₋₆ alkyl, C₂₋₆ alkenyl, aryl, C₁₋₄ alkyl aryl, heteroaryl, C₁₋₄ heteroaryl;
R⁵ and R⁶ are independently selected from the group consisting of: hydrogen, C₁₋₆ alkyl, C₁₋₆ haloalkyl, C₂₋₆ alkenyl, C₃₋₆ cycloalkyl, aryl, C₁₋₆ alkyl aryl, C₁₋₆ alkanoyl aryl, heteroaryl, C₁₋₆ alkanoyl heteroaryl, and C₁₋₆ alkyl heteroaryl; provided always that both R⁵ and R⁶ are not hydrogen;
R⁷ and R⁸ are independently selected from the group consisting of: H, C₁₋₄ alkyl and halo;
X is —(CR^{a}R^{b})ₘ(CR^{a}═CR^{b})ₙ(CR^{a}R^{b})ₒ where R^{a} and R^{b} are independently selected from the group consisting of: H, methyl, ethyl and halo and m, n, and o are independently 0, 1, 2, or 3 provided that m+n+o is not more than 3; and wherein
each of the above groups R¹ to R¹⁰ may, where chemically possible, be independently optionally substituted by from 1 to 5 groups chosen independently at each occurrence from the groups consisting of: halo, C₁₋₃ alkyl, halo C₁₋₃ alkyl, C₁₋₃ alkoxy, C₁₋₃ haloalkoxy, hydroxy, and cyano.

2. A compound of claim 1, wherein R¹ is C₁₋₆ alkyl.

3. A compound of claim 1, wherein R² is —CONR⁹R¹⁰ where R⁹ is H and R¹⁰ is aryl.

4. A compound of claim 1, wherein R³ is aryl.

5. A compound of claim 1, wherein R⁴ is optionally substituted aryl.

6. A compound of claim 1, wherein R¹ is i-propyl, R² is —CONHPh, R³ is phenyl and R⁴ 4-fluorophenyl.

7. A compound of claim 1, wherein R⁵ is selected from the group consisting of: hydrogen, aryl, C₁₋₆ alkyl aryl, C₁₋₆ alkanoyl aryl, heteroaryl, C₁₋₆ alkanoyl heteroaryl, and C₁₋₆ alkyl heteroaryl.

8. A compound of claim 7, wherein R⁵ is hydrogen.

9. A compound of claim 7, wherein R⁵ is selected from the group consisting of: —C₁ alkyl-Ph, —C₂ alkyl-Ph, —C₃ alkyl-Ph, and —C₄ alkyl-Ph.

10. A compound of claim 9, wherein R⁵ is benzyl.

11. A compound of claim 7, wherein R⁵ is C₁₋₆alkanoyl pyridine.

12. A compound of claim 11, wherein R⁵ is 3-methanoyl pyridine.

13. A compound of claim 1, wherein R⁶ is selected from the group consisting of: hydrogen, C₁₋₆ alkyl, C₁₋₆ haloalkyl, C₂₋₆ alkenyl, C₃₋₆ cycloalkyl, optionally substituted aryl and C₁₋₆ alkyl aryl.

14. A compound of claim 13, wherein R⁶ is selected from the group consisting of: C₁₋₆ alkyl, C₃₋₆ cycloalkyl, C₂₋₆ alkenyl and optionally substituted aryl.

15. A compound of claim 14 wherein R⁶ is selected from the group consisting of: methyl, ethyl, propyl, butyl, cyclohexyl and allyl.

16. A compound of claim 13, wherein R⁶ is optionally substituted aryl.

17. A compound of claim 16, wherein R⁶ is selected from the group consisting of: C₁₋₆ alkoxy substituted phenyl.

18. A compound of claim 16, wherein R⁶ is 2,4-dimethoxyphenyl.

19. A compound of claim 1, wherein R⁵ is hydrogen and R⁶ is an C₁₋₆ alkyl, C₃₋₆ cycloalkyl or an optionally substituted aryl.

20. A compound of claim 1, wherein R⁵ is an optionally substituted benzyl and R⁶ is an optionally substituted C₁₋₆alkyl or an optionally substituted C₂₋₆ alkenyl.

21. A compound of claim 1, wherein R⁵ is a C₁₋₆ alkanoyl heteroaryl and R⁶ is an optionally substituted C₁₋₆alkyl.

22. A compound of claim 1, wherein R⁷ is H and R⁸ is H.

23. A compound of claim 1, wherein each R^{a} is H, each R^{b} is H and m=2, n=0 and o=0.

24. A compound of claim 1 which has a structure selected from:

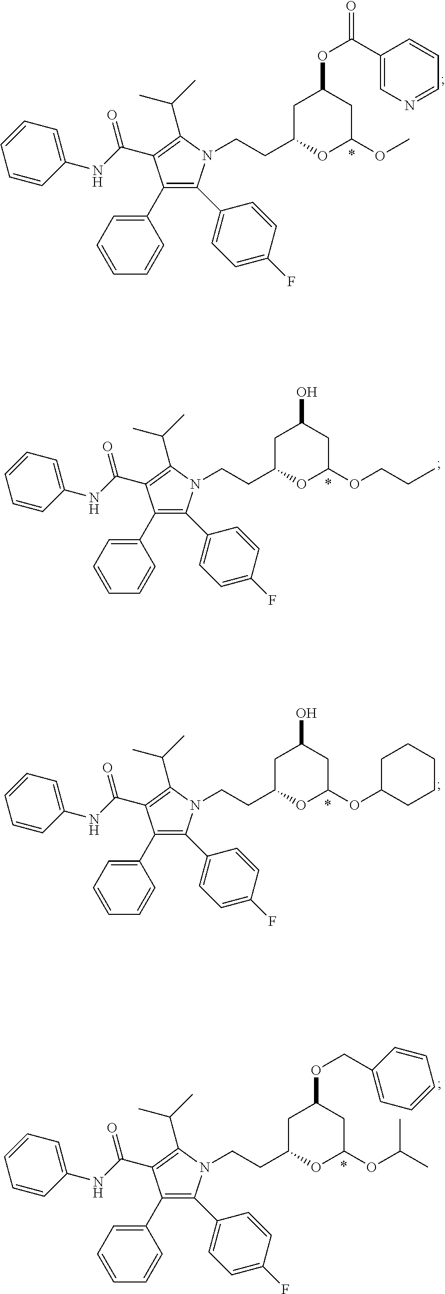

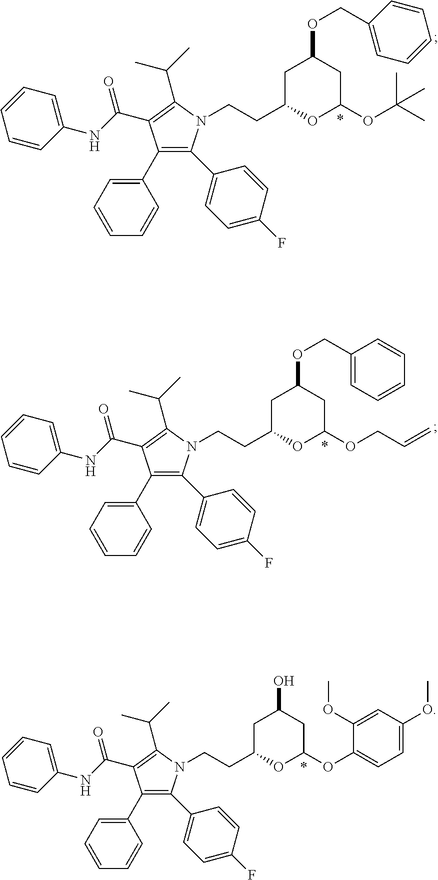

25-27. (canceled)
